(19) United States
(12) Patent Application Publication
(10) Pub. No.: US 2020/0261736 A1
(43) Pub. Date: Aug. 20, 2020

(54) MONOPOLAR RF SUBCUTANEOUS FAT TREATMENT SYSTEMS AND METHODS

(71) Applicant: Candela Corporation, Marlborough, MA (US)

(72) Inventors: Dany Berube, Milpitas, CA (US); Yossef Ori Adanny, Mitzpe Ilan (IL); Vladimir Goland, Ashdod (IL); Amit Sasson, Mishmarot (IL); Kevin Schomacker, Maynard, MA (US)

(73) Assignee: Candela Corporation

(21) Appl. No.: 16/783,264

(22) Filed: Feb. 6, 2020

Related U.S. Application Data
(60) Provisional application No. 62/807,541, filed on Feb. 19, 2019.

(51) Int. Cl. A61N 1/40; A61B 5/01; A61B 5/05; A61N 1/08

(52) U.S. Cl. CPC A61N 1/403; A61N 1/08; A61B 5/0507; A61B 5/01

(57) ABSTRACT

A subcutaneous fat treatment system and method. An electrode is for application to a patient's epidermis above subcutaneous fat to be treated. There may optionally be a cooling subsystem for cooling the electrode. A sensor such as a microwave radiometer measures the temperature of the subcutaneous fat to be treated. In addition or in the alternative, a subcutaneous fat thickness measurement may be used. A radio frequency source is for applying radio frequency energy to the electrode. A controller subsystem is responsive to the sensor and/or the subcutaneous fat thickness measurement and controls the radio frequency source to determine the thermal dose applied to the subcutaneous fat being treated and automatically adjust the radio frequency energy supplied to the electrode subjecting the subcutaneous fat to a thermal dose of between 0.1 and 10.0.

### RELATED APPLICATIONS

This application claims benefit of and priority to U.S. Provisional Application Ser. No. 62/807,541 filed Feb. 19, 2019, under 35 U.S.C. §§ 119, 120, 363, 365, and 37 C.F.R. § 1.55 and § 1.78, which is incorporated herein by this reference.

### FIELD OF THE INVENTION

This invention relates to monopolar radiofrequency (RF) subcutaneous fat treatment systems and methods.

### BACKGROUND OF THE INVENTION

Non-invasive bipolar RF systems have been known in the field many years. As example, US patent application 2006/0036300A1 disclosed a bi-polar chamber using non-invasive surface electrodes, along with a vacuum chamber, to elevate fat temperature and create volume reduction through apoptotic and/or necrotic adipocyte responses. Because of the physics involved, however, bi-polar RF modality is better at creating a thermal profile in the dermis instead of subcutis (fat). Indeed, the electrical conductivity of the dermis is about one order of magnitude higher in dermis than subcutaneous fat (0.27 versus 0.0267 S/m, respectively at 1 MHz) so the RF current has a tendency to stay within the dermal layer without going much through the subcutaneous layer. This situation is therefore adequate when a dermal response is desired for skin tightening treatment for example, but sub-optimal when fat treatment such as non-invasive fat removal is desired.

Invasive bipolar RF systems for fat reduction are also known. See, for example, U.S. Patent Application No. 2011/0046615 incorporated herein by this reference. In general, needles are inserted into the fat layer and supplied with radio frequency energy. This can allow a precise delivery of RF energy into the fat layer while preserving the dermal layer of the skin since the active parts of the needles are inserted directly in the fat layer. However, the needles inserted into the fat layer can result in discomfort and/or pain to the patient.

Non-invasive monopolar RF treatment methods of subcutaneous fat have also been proposed. See U.S. Published Application No. 2010/0211060 incorporated herein by this reference. In general, a monopolar system includes a console supplying control signals to a RF supply and the output from the RF supply is supplied to a hand piece electrode and optionally a return pad electrode. In general, the frequencies used range from 100 KHz to 10 MHz.

### SUMMARY OF THE INVENTION

One of the most challenging issues with non-invasive bipolar or monopolar RF energy delivering system is controlling the amount of energy or power delivered by the system to reach and/or maintain a precise desired temperature. Indeed, several physiological factors can affect the temperature profile in the fat layer and alter the levels of temperature in fat. For example, the electrical conductivity of fat can vary from patient to patient, and so too can the fat thickness, the blood perfusion levels, the thermal conductivity, the heat capacity, and the like. Without a way to measure fat temperature in real time during RF energy application, it is very difficult to properly control the energy application to predictably and consistently reach and/or maintain proper temperature levels.

As one way to circumvent the issue, inserting temperature sensors embedded within a hollow needle into the treatment zone as a way to monitor the temperature in situ have been proposed and used. See Franco, W, Kothare A, and Goldberg D. J., Controlled Volumetric Heating of Subcutaneous Adipose Tissue Using a Novel Radiofrequency Technology, Lasers Surg Med, 2009; 41:745-750. Although the technique could be effective, inserting needles in fat during treatment can be painful and undesirable.

In addition, the effectiveness of a thermal treatment for biological tissue is usually linked to the concept of a thermal dose, which is a combination of time and tissue temperature—not tissue temperature alone. Clinical investigations have shown that delivering a thermal dose of less than about 0.1 in the subcutis resulted in very subdued biological response, while delivering a thermal dose of more than about 10 resulted in undesirable permanent scar-based tissue such as nodules. These observations further establish the need for a system capable of measuring the subcutaneous temperature during thermal treatment in a non-invasive way.

The following document discloses using a microwave radiometer to monitor the subcutaneous temperature during a thermal treatment and to calculate a thermal dose in the subcutis in order to deliver therapeutically effective levels of thermal energy, so clinical results can be optimized without adverse events. The capability of the microwave radiometer to measure the subcutaneous temperature can also be used as a feedback system which, along with a temperature control algorithm, can control the subcutaneous tissue temperature to reach and maintain a selected or pre-defined target temperature.

The use of non-invasive technologies for fat removal or fat treatment without temperature measurement system can lead to inconsistent clinical results. In addition, using invasive technologies such as RF needles can be painful and requires additional skills from the users, and requires additional restrictions during treatment such as the need for a sterile field and devices, or the need to manage pain with diluted lidocaine or tumescent fluid injection for example.

Furthermore, the inventors hereof have discovered that if the thermal dose applied to the treatment area is too low, fat reduction is minimal or non existent. Conversely, if the thermal dose applied to the treatment areas is too high, palpable lumps due to inflammation of the subcutaneous fat are observed.

Featured, in one preferred aspect, is a monopolar RF subcutaneous fat treatment system and method wherein a non-invasive microwave radiometer is used to determine the temperature profile of the dermis and subcutaneous fat and the thermal dose during treatment without having to insert temperature probes into the patient's body to take direct temperature measurements. The microwave radiometer may be used to measure the subcutaneous fat temperature and the thermal dose delivered to the subcutaneous fat to ensure adequate therapeutic thermal dose in fat was delivered, without creating undesirable permanent nodules or other adverse event.

Also featured is a method of measuring a subcutaneous fat thickness, and using a calibration curve to select the output power for the energy source in order to reach a desired maximal subcutaneous temperature.

Featured is a subcutaneous fat treatment system and method comprising an electrode for application to a patient's epidermis above subcutaneous fat to be treated. A sensor, in one embodiment, preferably, a microwave radiometer, measures the temperature of the subcutaneous fat to be treated. Optionally, a subcutaneous fat thickness measurement is also taken. A radio frequency source applies radio frequency energy to the electrode. A controller subsystem is responsive to the sensor and/or the subcutaneous fat thickness measurement and controls the radio frequency source. The controller subsystem may automatically adjust the radio frequency energy supplied to the electrode and may subject the subcutaneous fat to a thermal dose of between 0.1 and 10.0. The controller subsystem may adjust the radio frequency energy supplied to the electrode to subject the subcutaneous fat to a temperature of between 40° C. and 50° C. and, in one embodiment, for between 10 and 30 minutes.

In one design, the system further includes a cooling subsystem for cooling the electrode. The cooling subsystem may include a channel in the electrode for a cooling fluid. The microwave radiometer may also be configured to further measure the temperature of the patient's epidermis. One preferred radiometer includes an antenna associated with the electrode. In one example, the antenna is printed on the electrode surface contacting the patient's epidermis. In another example, the electrode includes an opening and the antenna is located in the opening. Further, the electrode may include an electrically non-conductive periphery material to limit the electrode patient contact surface.

Preferably, the controller is configured to determine the thermal dose by calculating the thermal dose as a function of the temperature of the subcutaneous fat being treated and the length of time the radio frequency energy is supplied to the electrode. In addition, or in the alternative, the controller determines the thermal dose and function of the measured subcutaneous fat thickness.

Also featured is a method of treating subcutaneous fat. One preferred method comprises applying an electrode to a patient's epidermis above subcutaneous fat to be treated, measuring the temperature of the subcutaneous fat to be treated preferably by using a microwave radiometer and/or optionally measuring the thickness of the subcutaneous fat. Radio frequency energy is applied to the electrode and the thermal dose applied to the subcutaneous fat being treated is determined and the radio frequency energy supplied to the electrode is adjusted based on the measured temperature of the subcutaneous fat being treated and/or the thickness of the subcutaneous fat and the subcutaneous fat is subjected to a thermal dose of between 0.1 and 10.0.

Also featured is a subcutaneous fat treatment system and method. An electrode is applied to a patient's epidermis above subcutaneous fat to be treated. There is preferably a cooling subsystem for cooling the electrode. A subcutaneous fat thickness measurement system measures the thickness of the subcutaneous fat. A radio frequency source for applies radio frequency energy to the electrode. A controller subsystem is responsive to a measured subcutaneous fat thickness and controls the radio frequency source and the cooling subsystem and is configured to determine the thermal dose applied to the subcutaneous fat being treated, and automatically adjusts the radio frequency energy supplied to the electrode based on the measured thickness of the subcutaneous fat being treated and subjecting the subcutaneous fat to a thermal dose of between 0.1 and 10.0. Also featured is a subcutaneous fat treatment system comprising an electrode for application to a patient's epidermis above subcutaneous fat to be treated and a sensor for measuring the temperature of the subcutaneous fat to be treated. A radio frequency source applies radio frequency energy to the electrode. A controller subsystem, is responsive to the sensor and controls the radio frequency source and is configured to control the radio frequency source to apply radio frequency energy to the electrode to reach and maintain a desired set subcutaneous fat temperature, determine the thermal dose applied to the subcutaneous fat being treated, and automatically adjust the radio frequency energy supplied to the electrode based on the measured temperature of the subcutaneous fat being treated and subjecting the subcutaneous fat to a thermal dose of between 0.1 and 10.0.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram showing the primary components associated with a prior art non-invasive bi-polar RF treatment apparatus;

FIG. 2 is a schematic view showing the primary components associated with an exemplary monopolar subcutaneous fat treatment system;

FIG. 3A is a circuit equivalent diagram of a non-invasive bi-polar system;

FIG. 3B is a circuit equivalent diagram of a non-invasive monopolar system;

FIG. 4 is a schematic view showing two cooled radio frequency electrode assemblies used in an example of the subject invention;

FIG. 5 is a schematic view of an example of a RF electrode assembly;

FIG. 6 is an exploded view of the RF electrode assembly of FIG. 5;

FIG. 7 is a schematic view showing the bottom of the RF assembly of FIGS. 5 and 6;

FIG. 8 is a schematic view showing an exemplary cooling channel for the RF electrode assembly;

FIG. 9 is a block diagram showing an example of one preferred monopolar subcutaneous fat treatment system in accordance with the invention;

FIG. 10 is a flow chart depicting the primary method of treating subcutaneous fat and also depicting the computer's instructions associated with the controller subsystem of in FIG. 9;

FIG. 11 is a graph showing the maximal area of adipocyte damage versus thermal dose;

FIG. 12 is a chart showing the incidence of lumps for different thermal dose thresholds;

FIG. 13 is a graph comparing the thermal dose to the maximal measured temperature including instances where palpable lumps were detected;

FIG. 14 is block diagram showing the primary components associated with an exemplary monopolar subcutaneous fat treatment system in accordance with the invention;

FIGS. 15A-15C show examples of a microwave radiometer antenna located within the perimeter of a RF electrode in accordance with the subject invention;

FIG. 16 is another view of the bottom surface of an electrode including a microwave antenna in accordance with aspects of the invention;

FIG. 17 is a view of the thermal profiles in the dermis and subcutis using a cooling circulating water temperature of 20° C. at various power levels; and

FIG. 18 is a graph showing power levels required to reach maximal subcutaneous temperature of 46 and 48° C., as a function of the subcutaneous fat thickness.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

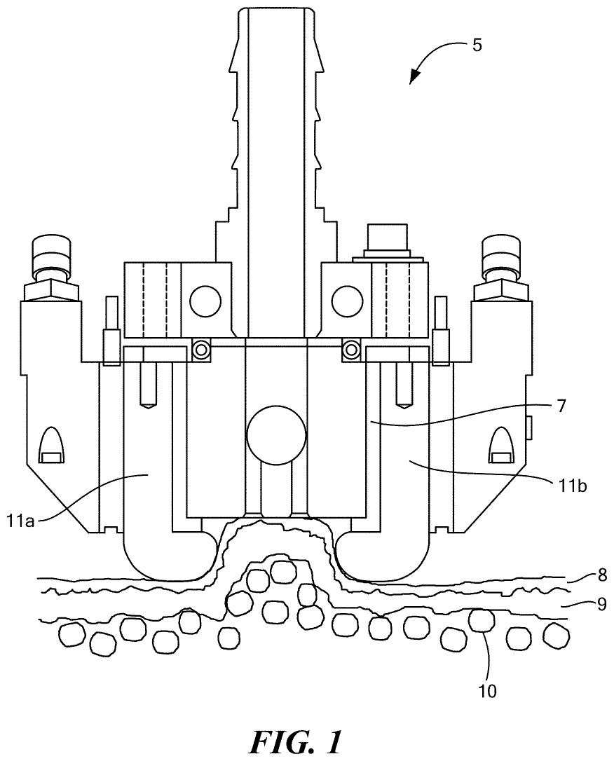

FIG. 1 shows an example of a non-invasive bi-polar RF treatment system where applicator 5 is shown for applying negative pressure and RF energy to skin in accordance with one embodiment of the invention. The applicator is configured to be connected to an RF generator (not shown). The applicator is configured to be applied to a region of the skin of an individual to be treated. The applicator includes an applicator body formed from a material having a high electrical conductivity and enclosing a bell-shaped chamber. The bell-shaped chamber is open on the bottom so that when applied to a region of skin, the skin is in contact with the bell-shaped chamber. The skin tissue includes an epidermal layer 8 and a dermal layer 9 overlaying a layer of subcutaneous adipose tissue 10.

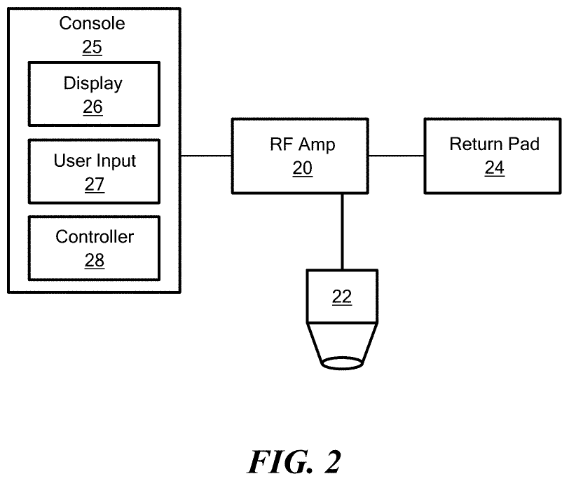

FIG. 2 shows a non-invasive monopolar radio frequency treatment system where RF energy from source 20 is applied to electrode 22 to be placed on the patient's skin surface (epidermis) and optionally to return pad electrode 24 which may be located at the patient's back. Console unit 25 may include a display 26 and a user input region 27 such as touch screen display. Controller 28 including a computer processor controls the entire system such as by controlling the signals to RF supply 20.

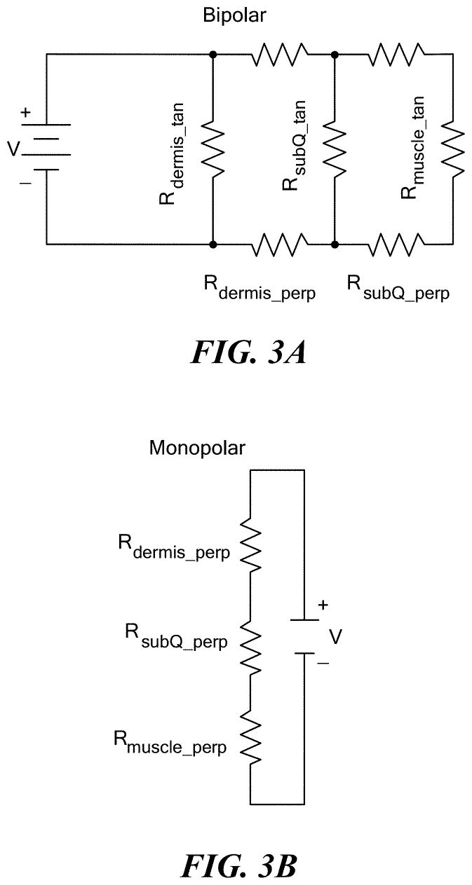

FIGS. 3A-3B depict the difference between bi-polar and monopolar non-invasive RF treatment systems. In a bi-polar system (FIG. 3A), the electrical current takes the path of least resistance through the dermis and energy is primarily absorbed in the dermis. In a monopolar system, in contrast, (FIG. 3B), the electrical current is forced to go through all the tissue layers and energy is primarily absorbed in the layer of highest resistivity, which is the subcutaneous fat layer.

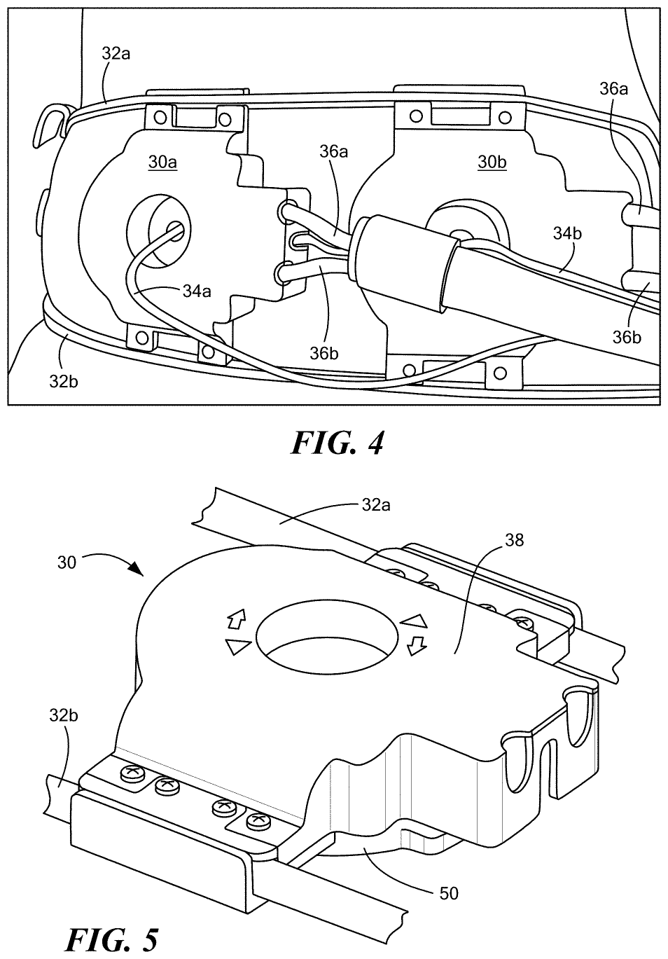

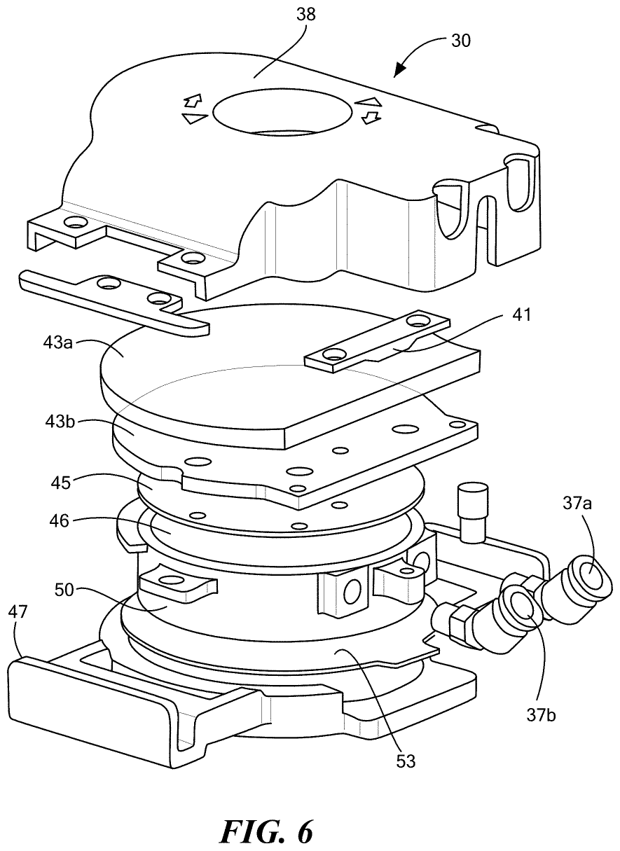

FIG. 4 depicts two exemplary monopolar RF electrode assemblies 30a, 30b applied to a patient's abdominal area to treat subcutaneous fat below the dermis. Bands 32a, 32b keep the electrode assemblies in place during treatment. RF energy is supplied to the electrode assemblies 34a, 34b from an RF source via electrical cables 34a, 34b. The electrode assemblies were cooled via a cooling subsystem including fluid (e.g., a cooling liquid such as water) flowing an input and return line sets 36a and 36b. An exemplary electrode assembly 30, FIGS. 5-6 includes top cap 38, electrical connector 41, caps 43a, 43b, gasket 45, electrode cavity 46, belt housing 47, and electrode 50.

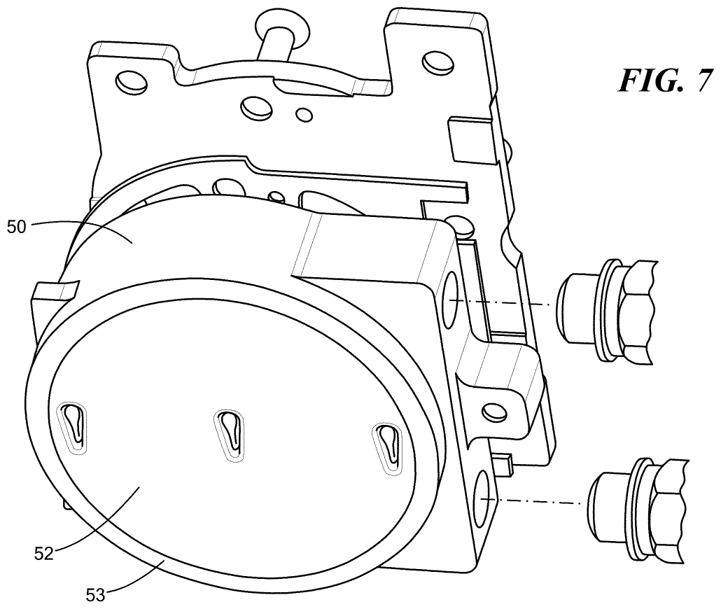

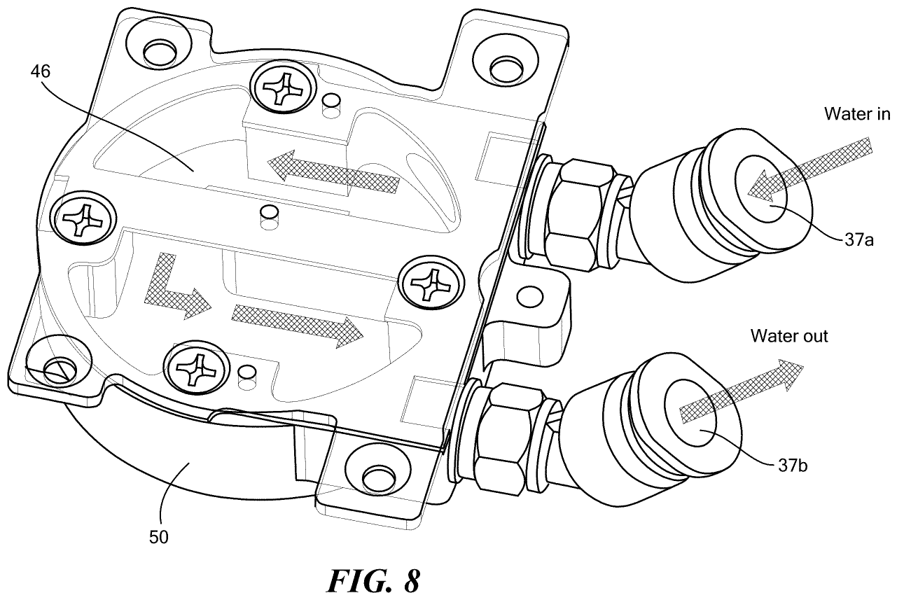

As shown in FIG. 7, the electrode 50 has a surface 52 for contacting the patient's dermis. Electrode 50 is preferably made of an electrically conductive material, such as a metal, which may be copper with a hard chromium coating as one example. FIG. 8 shows the cooling channel 46 inside the electrode housing including a fluid inlet 37a and fluid outlet 37b to cool electrode 50 to prevent damage to the patient's dermis. The cooling fluid could be water, saline, glycol, or any other fluid typically used in cooling system. Cooling channel 46 is preferably a part of the electrode cooling system which in addition includes a Liquid Chiller Module (LCM) which is used to keep the liquid at a desired temperature, (e.g., 20-40° C.). The LCM could be a commercially available system such as model FP00039 from Aspen System (Marlborough, Mass.). The temperature of the cooling fluid can be fixed or adjusted before or during treatment A thin non-conductive electrically isolating material 53 such as mylar, Teflon, or polyimide as examples, may be placed on the electrode to keep the portion cooled and electrically inactive in order to thermally protect the area beyond the active electrode. Indeed, the area immediately beyond the electrically active part of the RF electrode has a tendency to heat up when monopolar RF energy is used, and it is important to keep this area cooled to thermally protect the skin surface and avoid adverse events such as burns, blisters, discolorations, and other non-desirable outcomes.

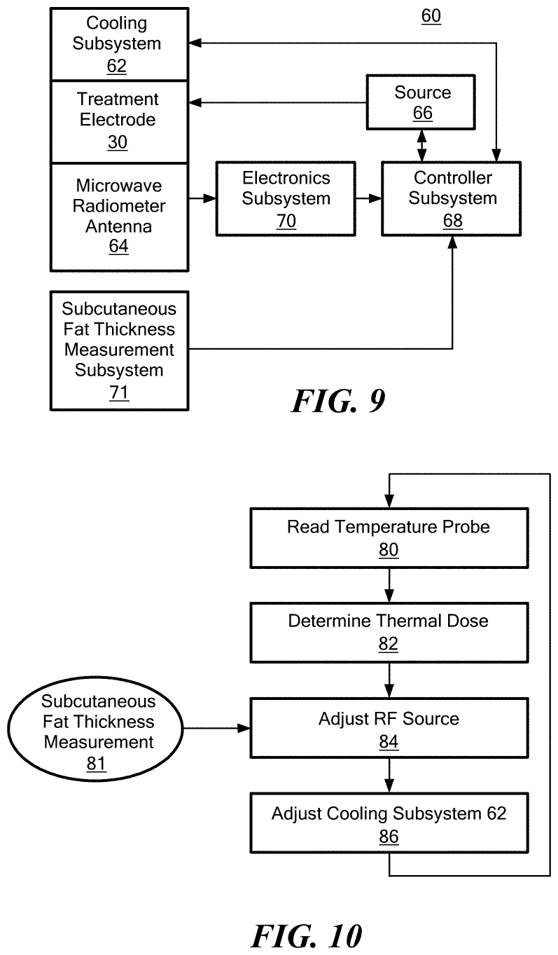

One exemplary system 60, FIG. 9 includes a treatment electrode 30, a cooling subsystem 62 associated with a treatment electrode and also a microwave radiometer antenna 64 associated with the system. In one preferred embodiment, the microwave radiometer antenna is printed on the face of the electrode which is in contact with the patient's epidermis. In another preferred embodiment, the microwave radiometer antenna is separated from the RF electrode and located within the periphery of the electrode. In yet another preferred embodiment, the metallic part of the microwave radiometer antenna is separated from the skin surface by a layer of dielectric. RF source 66 supplies RF energy to the treatment electrode and is controlled by controller subsystem 68 which also preferably controls cooling subsystem 62 to properly cool the electrode to prevent damage to the patient's dermis. The non-invasive microwave radiometer including antenna 64, an electronic subsystem 70 including a radiometer algorithm which provides, after calculation by the radiometer algorithm, the temperature profile of the dermis and/or subcutis as discussed infra. Radiometer algorithms capable of calculating temperature or thermal gradients have been disclosed in the literature and are beyond the scope of this document. See also U.S. Pat. Nos. 4,346,716 and 4,632,127 incorporated herein by this reference.

The controller and electronic subsystem for the microwave radiometer may be combined within the same module. Typically, a PC or equivalent is used to implement the radiometer algorithm, which is used to output one or several temperature value(s), or a thermal gradient in 1, 2, or 3 dimensions depending on the algorithm. Software instructions stored in memory and executed by one or more processors are configured to read and optionally display the temperature profile of the dermis and/or subcutis at the treatment area, step 80, FIG. 10 and determine the approximate thermal dose, step 82. Radio frequency source 66 FIG. 9 is thus preferably controlled, step 84, to subject the subcutaneous fat to a thermal dose of between 0.1 and 10.0 for maximum effect in this without the creation of permanent nodules or at least minimizing the incidence of permanent nodules and to minimize the incidence of temporary lumps while at the same time the cooling subsystem may be controlled, step 86, to prevent harm to the patient's dermis and/or patient discomfort. The cooling subsystem control may include controlling the volume of water supplied to the electrode housing, the temperature of the water, supplied to the electrode housing, the flow rate, or all three parameters. In some instances, it may be useful to increase the cooling fluid temperature above body temperature in order to warm-up the skin surface. In one example, warming up the fluid to about 44 to about 46° C. for about 20 minutes may be useful to improve the visual appearance and/or the quality of the skin and/or create skin tightening. For a skin tightening procedure in conjunction with the subcutaneous fat treatment, the fluid temperature may be adjusted to a higher temperature (e.g., 44° C.) and held constant. For a skin tightening procedure following the subcutaneous fat treatment, the fluid temperature may be set lower (e.g. 20° C.) during the subcutaneous fat treatment and following that higher (e.g., 44° C.) during the skin tightening procedure. Adjustments to the fluid temperature may be made during any treatment or the fluid temperature may be held constant. In addition, controlling the radiofrequency source typically includes controlling the radio frequency power and the duration of time the radio frequency power is supplied to the electrode.

The temperature information from a real-time temperature measurement of the subcutis can also be used to control the output power of the radiofrequency source in order to reach and maintain a desired subcutaneous target temperature. The temperature information can either be from a temperature probe invasively inserted in the subcutis, or from the measurement taken non-invasively by a microwave radiometer. In both case, a proportional-integral (PI) controller can be used to adjust the RF output power in real time. Using a PI controller (or any other form of controllers, such as a PID controller as an example), can be advantageous to make sure that a target subcutaneous temperature is reached and maintained to manage the discomfort levels during treatment, to insure that the minimal temperature is at least reached to insure efficacy, and to insure that a maximal temperature is not exceeded to ensure patient safety and minimize the risk of undesirable adverse events, such as the creation of a permanent nodule as one example.

In a preferred embodiment, the microwave radiometer would be optimized to measure the temperature of the subcutis, preferably in the area of the maximal subcutaneous temperature which is usually located between 5 and 15 mm underneath the subcutaneous junction when skin cooling of about 20° C. is applied by the electrode. The subcutaneous tissue temperature would then be used as the input to a controller, preferably a PI controller, capable of controlling the output power delivered to the energy applicator (one or more RF electrode(s) in the preferred embodiment), to reach and maintained a pre-selected target temperature (T_target), and to apply a safe and effective thermal dose in the subcutis to create a desired effect, the reduction subcutaneous volume, by evoking adipocytes apoptotic and/or necrotic responses in this example.

In a preferred embodiment, the PI controller would be programmed using the following equation (or any equivalent equation expressed in another mathematical form):

P=kₚ·ΔT+kᵢ∫ΔT·∂t (1)

### Where:

P is the output power;
ΔT is the difference between the target and the measured temperature (T_target−T_measured);
t is the time, and
kₚ and kᵢ are the PI controller coefficients.
In a preferred embodiment, the PI controller coefficients would be selected to reach target temperature of 44 to 49° C. within about 5 to 15 minutes to maximize patient comfort, and not to overshoot or oscillate around the target temperature (T_target).
The technique would preferably include controlling the subcutaneous temperature using the measurement of a microwave radiometer (or from a probe inserted in the subcutis in a less preferred technique), using a PI controller to control the output power level of a RF energy source 66 (or any other energy source capable of delivering energy in the subcutis to increase the subcutaneous temperature, such as a microwave or ultrasonic source as examples), precisely reaching and maintaining a desired set subcutaneous temperature, calculating the thermal dose received by the subcutis in real time, and continuing the treatment until the subcutis has received a desired thermal dose before stopping the RF treatment. In certain cases, the desired temperature in the subcutis may not be reached in which case the controller will continue treatments beyond the intended time in order to achieve the desired thermal dose. In other cases, higher than typical subcutis temperatures may be reached where the desired thermal dose is reached earlier than the intended time in which case the controller would terminate treatments at this earlier time. Variables that affect reaching the desired temperature include higher or lower levels of tissue blood perfusion beyond what is typical for most patients, and the patient's tolerance to the procedure limiting the maximum RF power.

Preferred are thermal dose levels which create an adipocyte, apoptotic, or necrotic response. Clinical investigations with histological characterization have been performed to document the subcutaneous fat response to different thermal dose levels and safety and efficacy levels were defined.

A series of cooled RF electrode prototypes were designed, assembled and used clinically to evaluate the safety and efficacy profile of the proposed technique. The design of the electrode is shown in FIGS. 4-8. A needle mounted with a distal temperature sensor was used to directly measure the subcutaneous fat temperature and was positioned along the mid axis of the RF electrode. The needle was inserted in tissue prior to RF application in order to monitor the subcutaneous fat temperature in real time during treatment. The distal end of the needle was moved along the center line during treatment to locate and measure the maximal subcutaneous temperature during treatment. The objective was to measure the maximal subcutaneous temperature and to calculate the thermal dose received by the subcutis.

Multiple patients were treated in the abdominal area with the prototypes following proper ethic committee approval of the clinical protocol. Patients were followed for up at 1 and 3 months after the treatment where subcutaneous fat reduction was assessed underneath the treatment areas. The presence of lumps was also assessed at all follow-up visits.

Histology samples for patients already scheduled for abdominoplasty were also collected after a RF treatment. The collected samples were stained with H&E to document the wound response and observe evidences of adipocytes necrosis for different thermal dose values.

The thermal dose Ω was calculated as follow:

Ω(t)=A·∫e^{(-EJ}_{R·T(t))}∂t (2)

where:
t is the time,
Ω(t) is the thermal dose,
T(t) is the temperature,
A is a constant representing the molecular collision frequency which is assumed to be unchanged over the temperature range, and equal to 2.19×10¹²⁴ s⁻¹,
E is the heat of inactivation, and is equal to 777600 J/mol, and
R is the molar gas constant, equals to 8.314 J*K⁻¹*mol⁻¹.

It is to be noted that the E and A parameters shown above are generic parameters for a variety of soft biological tissue and not specific to subcutaneous fat. It may be possible to further and better characterize these parameters for fat, which could then be used to calculate the thermal dose Ω using the equation 1 above.

Histology samples were taken from two patients for a total of 8 treatment locations. The delivered thermal dose ranged from 0.03 to 3.21. At the lower end of the tested range (Ω=0.03) there was almost no histologic evidence of thermal injuries.

Another histology example for a mid-range thermal dose of 1 resulted in areas of adipocyte damage, with the largest observed sample covering an area of about 11.2 mm².

In all treated areas, the largest thermal dose value delivered in subcutis was 3.53. On the treated area, the patient reported a nodule prior to tissue harvesting days after the procedure. Histological assessment revealed broad areas of fat necrosis in an area covering approximately 210 mm² in the histological sample, which accounted for about 60% of the sampled area.

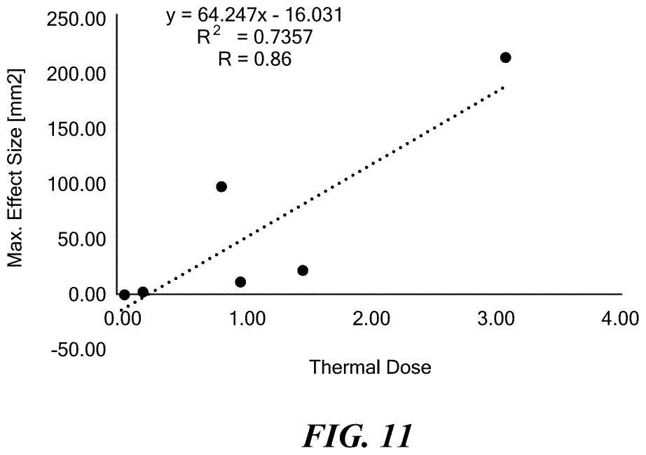

The maximal histologically observed areas of adipocyte damage vs. the measured delivered thermal dose was plotted in FIG. 11. Although the source data is limited, there seems to be a correlation between the two variables—the size of the thermal injury increasing with the thermal dose.

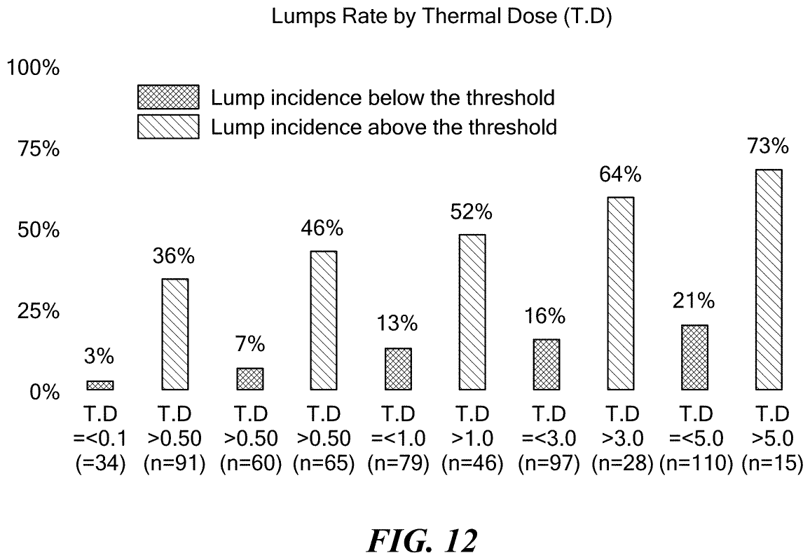

A total of 125 abdominal areas were treated with the prototypes. The presence of palpable lumps was assessed during all follow-up visits at 1 and 3 months post-treatment. The results are shown in FIG. 12 where the incidence of lumps was assessed against the delivered thermal dose. In general, the results showed an increased incidence of lumps with the thermal dose. Taking the results on the left as an example, 3% and 36% of the samples having received a thermal dose of less than 0.1 (n=34) and more than 0.1 (n=91), respectively, have resulted in palpable detection of lumps at follow-up.

The lumps are generally considered as transitory in nature and are signs of panniculitis, which is an inflammation of the subcutaneous fat. This effect was expected and is a sign that an inflammatory response have started to clear the necrotic fat, which has received sufficient thermal dose to bring adipocytes out of their viable range. However, lumps could lead to permanent nodules if the volume and extend of thermal injury could be sufficiently large to prevent a complete clearance by the wound healing sequences. In such case, a permanent nodule would be considered an adverse event.

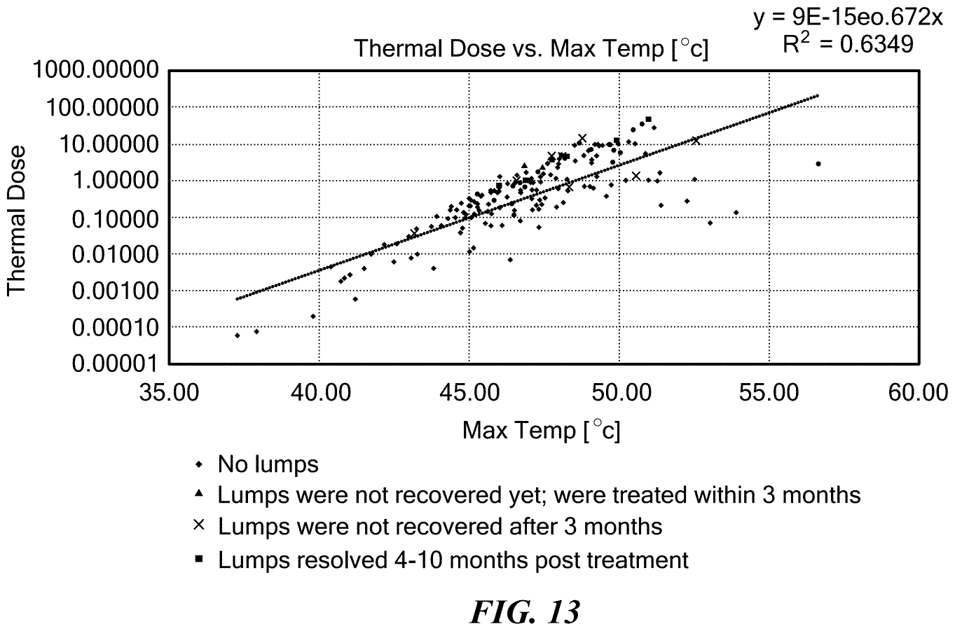

Similar data is presented in FIG. 13 where incidences of temporary lump detection (the green dots), incidence of permanent nodules (pink dots), and absence of temporary lump (blue dots) are presented in a Thermal Dose vs. Maximal Subcutaneous Temperature graph. The results showed that the incidence of lumps, or panniculitis, is negligible when the thermal dose, in a preferred setting, is below about 0.3 and significant when the thermal dose is above about 0.3. Since panniculitis is a desired effect, it is concluded that a thermal dose above about 0.3 is necessary to ensure efficacy. Conversely, still in a preferred setting, a dose above about 3 is expected to be too high and could produce undesired permanent nodules. In a less preferred setting, a thermal dose in the subcutis between 0.1 and 10 would be desired. Similarly, in a most preferred setting, the clinical results suggests that the maximal subcutaneous temperature should be kept between about 46 and 48° C. to optimize the efficacy and comfort levels, and minimize the safety concerns. It is worthwhile noting that tissue temperatures of more than about 50° C., for both dermis and subcutis, can trigger a nociceptive response and create intense pain for the patient, which is not desirable. In a less preferred setting, the maximal subcutaneous temperature should be kept between about 44 to about 50° C. Therefore, the clinical results suggest that the optimal thermal dose for a reasonable safety/efficacy profile for fat treatment would be between about 0.1 and about 10.

The overall objective of the clinical study was to reduce subcutaneous fat volume underneath the treatment areas. To assess the results, the subcutaneous fat thickness was assessed against baseline at different post-procedural follow-up timeframes. At 1-month follow-up, the thickness decreased was 1.79±1.52 mm (n=49), which increased to 2.37±2.37 mm (n=18) three months after treatment. The early results suggest that the treatment could be effective for reducing undesired subcutaneous fat.

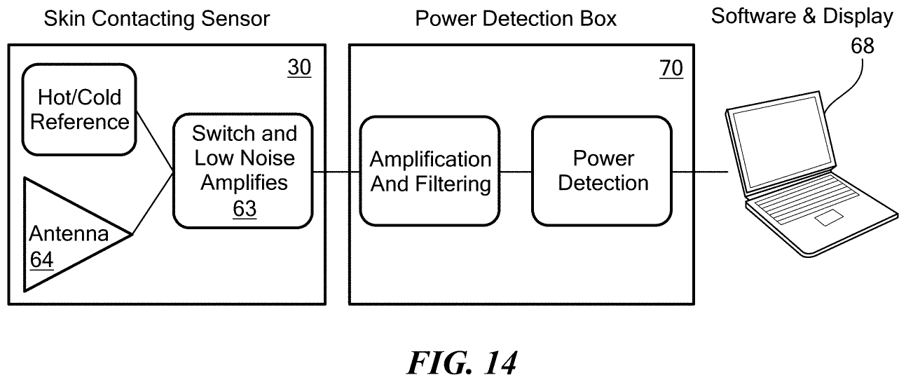

The usage of a microwave radiometer to measure the temperature of biological tissues or tissue-equivalent media (phantoms) have been described in the prior art. In general, a radiometer is designed to measure the level of electromagnetic (EM) emission in the microwave band, from about 500 MHz to about 10 GHz for example. A bloc diagram of a basic microwave radiometer system is shown in FIG. 14. The system generally comprises a skin contacting sensor 30, a power detection box 70, and a software and display with Graphical User Interface (GUI) 68. A microwave antenna 64 is used in the skin contacting sensor 30 to measure the microwave emission from the tissue of interest. To calibrate the microwave radiometer, at least one temperature reference is used, and preferably two temperature references: generally and preferable a first reference temperature below and a second reference temperature above the temperature range to be measured. For calibration purposes, the radiometer reads the microwave levels of at least one and preferably two reference temperature(s) to establish a relationship between the temperature and the microwave emission levels emitted by these reference temperature loads. In some instances reported in the literature, a short-circuit load is also used for calibration purposes in order to compensate for the undesirable effects of the EM noise within the system. A controllable switch 63 is used to feed the amplifier stages with the measured EM signal. Since the power levels of the measured signals are very low (typically in the order of a few picoWatts), a low noise amplifier 63 is mounted close to the antenna to elevate the power level of the EM signal to be transmitted to the power detection box 70 via a waveguide, typically a coaxial cable, before being amplified, filtered, and detected within the power detection box 70. Then, the software of PC 68 will treat the information to calculate the corresponding tissue temperature of the tissue being measured.

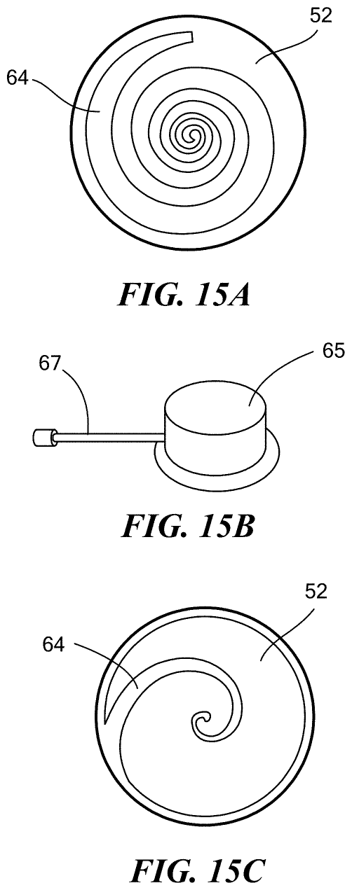

The microwave antenna used in the radiometer is preferably a broadband and directive microwave antenna with a small footprint. In FIG. 15A, a spiral antenna 64 is printed on a dielectric substrate 52, and mounted in a cylindrical metallic cavity 65, FIG. 15B, fed by a 50 ohm coaxial cable 67. In general, a layer of dielectric will be positioned between the antenna shown in FIG. 15A and the tissue surface in order to protect the metallic part 64 of the antenna and improve the matching between the tissue and the antenna and to therefore minimize the undesirable parasitic reflection at the antenna-tissue interface. FIG. 15C shows another antenna design.

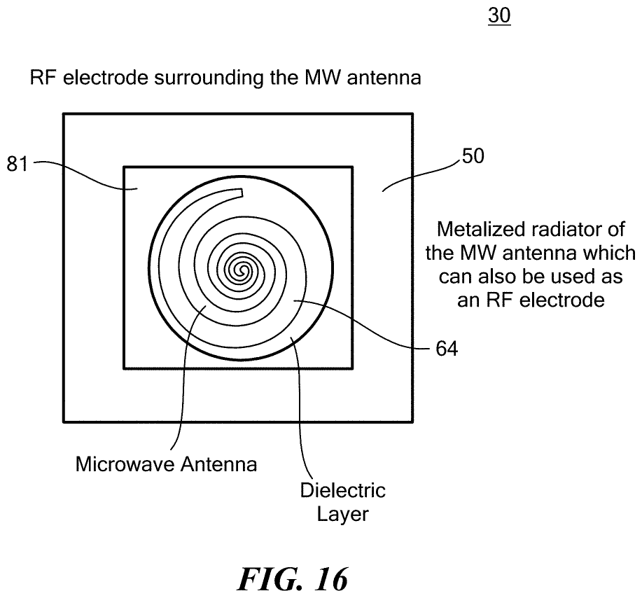

As explained earlier, one aspect of the inventions described herein is to use a microwave radiometer to non-invasively monitor the subcutaneous temperature levels during RF energy deposition (or other type of energy deposition such as microwave or ultrasound). To do so, the microwave antenna 64 used by the skin contacting sensor 30 of the microwave radiometer depicted in FIG. 14 should be located within the boundaries of the RF electrode, in order for the radiometer to be capable of sensing the subcutaneous temperature underneath the RF electrode where the tissue is being heated as a result of the energy deposition. FIG. 16 shows an example of an RF electrode assembly 30 incorporating the microwave antenna assembly 64 used by the microwave radiometer, where the microwave antenna 64 is located within the inner boundary of the RF electrode 50. In FIG. 16, an opening 81 is created within the outer boundaries of the electrode to accommodate the microwave antenna. Although the opening depicted in FIG. 16 is square, other shapes could be used—ideally the same shape as the microwave antenna, which is circular in FIG. 16. In case of circular microwave antenna shape, the diameter of the opening within the boundaries of the RF electrode in general needs to be equal or larger than the outer diameter of the microwave antenna. The location of the opening to receive the microwave antenna could ideally be located at the center of the RF electrode, or at an offset position. One factor to consider in the design and assembly of the microwave antenna and the RF electrode is to locate the microwave antenna in such a way that it can receive microwave emission from the tissue which is heated by the RF electrode, so a temperature change can be measured by the radiometer. As such, the microwave antenna assembly could be located outside the boundary of the RF electrode and, in one embodiment, angled in such a way that it would be capable of detecting such microwave emission and measure an equivalent temperature change. In another embodiment, the microwave antenna assembly could be located between two adjacent RF electrodes to monitor the tissue temperature in between.

The information measured by the microwave antenna of a radiometer can be used to calculate the deep tissue temperature, the subcutis in a preferred embodiment. Measuring the microwave emission in a single frequency band usually leads to one weighted average temperature in tissue, as the output of the radiometer algorithm. In addition, a thermal gradient in a perpendicular plane underneath the energy applicator, a RF electrode in the preferred embodiment, can be measured (or mathematically recreated) in real-time using a multi-frequency band radiometer. This allows to generate much more information about the temperature and/or thermal dose profiles which are generated underneath the energy applicator. As an explanatory note, the thermal dose Q defined in above sections can be calculated from a temperature profile within the perpendicular plane underneath the energy applicator, outputted by the microwave radiometer and temperature reconstruction algorithms implemented in the radiometer algorithm.

Further describing the concept when using a multi-frequency band radiometer, a thermal profile could be displayed on a Graphical User Interface (GUI) in order to allow a user to visualize in real time the temperature profile, in one embodiment, in a plane perpendicular to the energy applicator, which includes the subcutis in the preferred embodiment. The displayed thermal profile could also track down the maximal measured temperature value to insure comfort and safety. Similarly, a thermal dose (Ω) profile could be generated using the equation above since the temperature information is available within the described plane. The displayed thermal dose could also track down the maximal thermal dose value to insure safety and avoid adverse events such as the production of permanent scar-tissue nodule. The GUI could also display iso-contour lines for the thermal dose to evaluate the area or volume bounded by an iso-contour line of equal thermal profile. This would allow to define how much area or volume of subcutis has received a therapeutic thermal dose sufficient to generate a desired outcome such as a subcutaneous volume reduction.

As described in above section, the output power of an energy source connected to an applicator can be controlled to reach and maintain a desired tissue temperature. When a single frequency band radiometer is used, along with a PI controller (or equivalent), the weighted average temperature in tissue outputted by the radiometer can be used as the feedback measured temperature “T_measured” in the PI control equation shown in a previous section above. When a multi-frequency band radiometer is used, the feedback measured temperature can be selected at a spatial location anywhere within the plane of the reconstructed thermal gradient. In the case where there is a known location of preferred heating (commonly known as a “hot spot”), the location of preferred heating could be selected as the feedback location from which the temperature can be measured and used as the measured temperature (T_measured) in the PI controller equation above. Also, since many temperature values can be selected at different spatial location within the 2D plane or 3D volume, it is would be possible to modify the temperature feedback point during treatment in order to track the maximal temperature within the measurement plane or volume.

In some instance, it might not always be possible to use an invasive or non-invasive temperature sensor to provide real-time temperature measurement of the subcutis during RF treatment. In such instances, it would be desirable to use another technique which would allow reaching a desired maximal subcutaneous temperature, without temperature measurement feedback in the subcutis. In still another aspect of the invention, a calibration curve is used to select the proper amount of output power of the RF energy source (or other type) based on the thickness of the subcutaneous layer to be treated. Indeed, thicker layers of subcutis require more power to reach the same maximal temperature since there is more tissue to be heated.

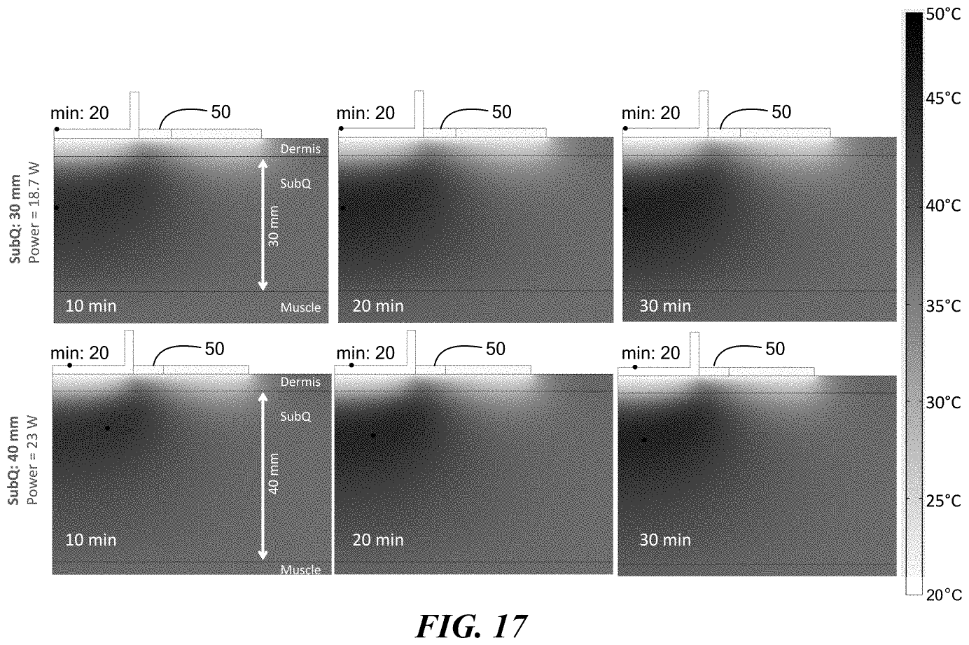

The power needed to elevate the subcutaneous temperature to a desired level needs to be established for different subcutaneous fat thickness conditions. To do so, a simulation technique using Finite Element Analysis (FEA) can be used to model the RF electrodes and the different tissue layers, to establish the relationship between the maximal subcutaneous temperature and the subcutaneous thickness. Commercially available FEA software package such as Comsol can be used to draw the electrode and tissue model, assign electrical and thermal properties of the different domains, assign the boundary conditions, and perform the simulations. FIG. 17 illustrates results examples obtained by simulating the electrode configuration of FIGS. 7 and 8. The thermal profiles are shown in the dermis and subcutis using a cooling circulating water temperature of 20° C. for two different subcutaneous thicknesses (30 and 40 mm). The target maximal subcutaneous temperature was 46° C. after 30 min of constant RF power delivery. It took 18.7 and 23.0 W to reach a maximum of 46° C. in the subcutis for a subcutaneous fat thickness of 30 and 40 mm, respectively. Performing several simulation runs to expending these results to several subcutaneous fat thicknesses, the relationship between power, subcutaneous fat thickness, and maximal subcutaneous temperature can be established.

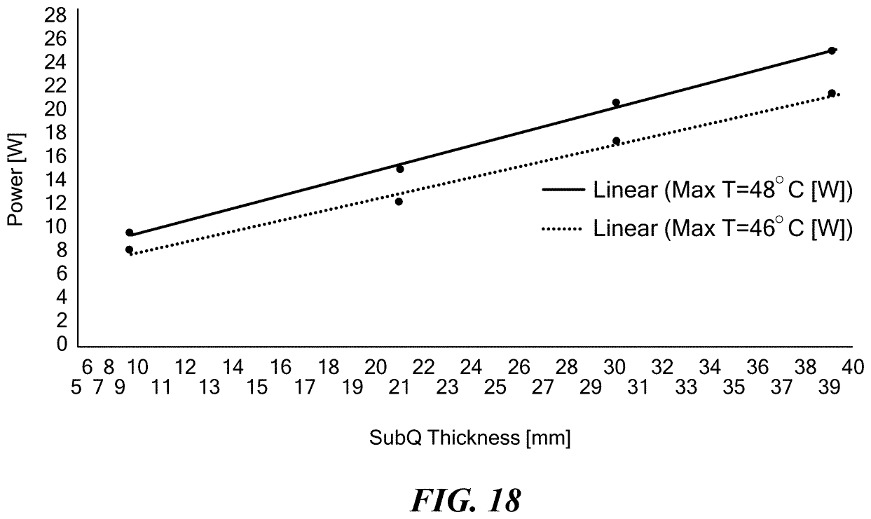

An example of such relationship obtained using the FEA simulation technique above is shown in FIG. 18. The relationship between the subcutaneous fat thickness (horizontal axis) and the required power (vertical axis) is displayed for two different maximal subcutaneous temperatures (46 and 48° C.). The simulations were performed by simulating a circular electrode area of 900 mm² with a fixed thermal boundary condition of 20° C. to simulate the effect of 20° C. circulating water within the electrode cavity, as shown in FIG. 8. The temperature results of FIG. 18 were obtained after 30 min of simulated RF energy deposition at constant power. To generalize the results, the power levels shown in the vertical axis could be normalized by dividing the values by the surface area of the simulated electrode (900 mm² in this case) and be expressed in power density (W/mm²), which could be generalized to different electrode area, all other variables being the same.

The method therefore preferably includes a subcutaneous fat measurement subsystem 71, FIG. 9, for measuring the subcutaneous fat thickness. An ultrasonic probe, a caliper, a Magnetic Resonance Imaging (MRI) system, or any other suitable method may be used. The subcutaneous thickness measurement could be performed manually or automatically prior to the subcutaneous fat treatment procedure, or could be performed by the same platform or device used to deliver the energy in the subcutis, assuming a proper subcutaneous fat thickness measuring system would be integrated in the device. A diagnostic ultrasonic probe could be integrated in the system to take the subcutaneous fat thickness measurement for example. Then, knowing the subcutaneous fat thickness, an operator could manually select the proper output power (or power density) levels to achieved a desired maximal subcutaneous temperature. Similarly, the power (or power density) vs. subcutaneous fat thickness relationship can be programmed in controller subsystem 68 in order to automatically select the power (or power density) after the subcutaneous fat thickness measurement result is entered as an input step 81, FIG. 10. An operator could manually enter the fat thickness result using a GUI interface. As another approach, the system could automatically receive the thickness value from the subcutaneous fat thickness measurement device, and automatically calculate the proper amount of power (or power density) to reach a pre-determined maximal subcutaneous fat temperature.

Thus controller subsystem 68 may be configured to adjust the radio frequency energy supplied to the electrode to subject the subcutaneous fat to a thermal dose of between 0.1 and 10.0 based on prior simulations as discussed above. The controller subsystem may further be configured to adjust the radio frequency energy supplied to the electrode to subject the subcutaneous fat to a temperature of between 40° C. and 50° C. In one embodiment, the controller subsystem is further configured to supply radio frequency energy to the electrode for between 10 and 30 minutes.

In FIG. 17, an inverse thermal gradient is obtained when the electrode temperature is lower than the maximum temperature in the subcutis. This means that the dermal temperature is lower than the maximal subcutaneous temperature. In yet in another aspect of the invention, the electrode temperature is kept at temperature below the target maximal subcutaneous temperature, and at a temperature low enough to allow comfortable treatment and protect against unwanted thermal injuries in dermis such as blister, burn, or scar. In a preferred embodiment, the electrode temperature is kept a temperature of 20° C. and the RF output power of the energy source adjusted to reach maximal subcutaneous temperature more preferably between 46 and 48° C., and preferably between 40 and 50° C. The features described above can also be used in combination with what was described above, notably with the PI controller to reach and maintain a desired subcutaneous temperature, and/or with a microwave radiometer to measure the subcutaneous temperature, temperature gradient, and thermal dose, in a non-invasive manner.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A subcutaneous fat treatment system comprising:
an electrode for application to a patient's epidermis above subcutaneous fat to be treated;
a microwave radiometer for measuring the temperature of the subcutaneous fat to be treated;
a radio frequency source for applying radio frequency energy to the electrode; and
a controller subsystem, responsive to the microwave radiometer and controlling the radio frequency source and configured to:
determine the thermal dose applied to the subcutaneous fat being treated, and
automatically adjust the radio frequency energy supplied to the electrode based on the measured temperature of the subcutaneous fat being treated and subjecting the subcutaneous fat to a thermal dose of between 0.1 and 10.0.

2. The system of claim 1 further including a cooling subsystem for cooling the electrode.

3. The system of claim 2 in which the cooling subsystem includes a channel in the electrode for a cooling fluid.

4. The system of claim 3 in which the microwave radiometer is configured to further measure the temperature of the patient's epidermis.

5. The system of claim 1 in which the radiometer includes an antenna associated with the electrode.

6. The system of claim 5 in which the antenna is printed on the electrode surface contacting the patient's epidermis.

7. They system of claim 6 in which the electrode includes an opening and the antenna is located in the opening.

8. The system of claim 1 in which the controller is configured to determine the thermal dose by calculating the thermal dose as a function of the temperature of the subcutaneous fat being treated and the length of time the radio frequency energy is supplied to the electrode.

9. The system of claim 1 further including a subcutaneous fat thickness measurement subsystem.

10. The system of claim 1 in which the controller subsystem is configured to control the radio frequency source based on a subcutaneous fat thickness measurement.

11. The system of claim 1 in which the electrode has a patient contact surface and further including an electrically non-conductive material on a periphery of the patient contact surface to limit an electrically active portion of the patient contact surface.

12. A method of treating subcutaneous fat, the method comprising:
applying an electrode to a patient's epidermis above subcutaneous fat to be treated;
measuring the temperature of the subcutaneous fat to be treated using a microwave radiometer;
applying radio frequency energy to the electrode;
determining the thermal dose applied to the subcutaneous fat being treated; and
automatically adjusting the radio frequency energy supplied to the electrode based on the measured temperature of the subcutaneous fat being treated and subjecting the subcutaneous fat to a thermal dose of between 0.1 and 10.0.

13. The method of claim 12 further including cooling the electrode.

14. The method of claim 13 further including cooling the electrode with a fluid.

15. The method of claim 14 further including measuring the temperature of the patient's epidermis using the microwave radiometer.

16. The method of claim 15 further including automatically controlling the fluid based on the measured temperature of the patient's epidermis.

17. The method of claim 12 in which the radiometer includes an antenna associated with the electrode.

18. The method of claim 17 in which the antenna is printed on the electrode surface contacting the patient's epidermis.

19. The method of claim 12 in which determining the thermal dose includes calculating the thermal dose as a function of the temperature of the subcutaneous fat being treated and the length of time the radio frequency energy is supplied to the electrode.

20. The method of claim 12 further including determining the thickness of the subcutaneous fat being treated and adjusting the radio frequency energy supplied to the electrode based on a measured thickness of said subcutaneous fat.

21. The method of claim 12 further including electrically isolating the periphery of the electrode.

22. A subcutaneous fat treatment system comprising:
an electrode for application to a patient's epidermis above subcutaneous fat to be treated;
a subcutaneous fat thickness measurement system;
a radio frequency source for applying radio frequency energy to the electrode; and
a controller subsystem configured to adjust the radio frequency energy supplied to the electrode based on the thickness of the subcutaneous fat being treated.

23. The system of claim 22 further including a cooling subsystem including a channel in the electrode for a cooling fluid.

24. The system of claim 22 further including a sensor for measuring the temperature of the subcutaneous fat to be treated.

25. The system of claim 23 in which the sensor is a microwave radiometer.

26. The system of claim 25 in which the radiometer includes an antenna associated with the electrode.

27. The system of claim 26 in which the antenna is printed on the electrode surface contacting the patient's epidermis.

28. The system of claim 26 in which the electrode includes an opening and the antenna is located in the opening.

29. The system of claim 24 in which the controller is further configured to determine the thermal dose by calculating the thermal dose as a function of the temperature of the subcutaneous fat being treated and the length of time the radio frequency energy is supplied to the electrode.

30. The system of claim 29 in which the controller is further configured to subject the subcutaneous fat to a thermal dose of between 0.1 and 10.0.

31. The system of claim 22 in which the electrode has a patient contact surface and further including an electrically non-conductive material on a periphery of the patient contact surface to limit the electrically active portion of the patient contact surface.

32. The system of claim 22 in which the controller is further configured to adjust the radio frequency energy supplied to the electrode to subject the subcutaneous fat to a thermal dose of between 0.1 and 10.0.

33. The system of claim 22 in which the controller is further configured to adjust the radio frequency energy supplied to the electrode to subject the subcutaneous fat to a temperature of between 40° C. and 50° C.

34. The system of claim 22 in which the controller is further configured to supply radio frequency energy to the electrode for between 10 and 30 minutes.

35. A method of treating subcutaneous fat, the method comprising:
applying an electrode to a patient's epidermis above subcutaneous fat to be treated;
determining the thickness of the subcutaneous fat to be treated;
applying radio frequency energy to the electrode; and
adjusting the radio frequency energy supplied to the electrode based on the thickness of the subcutaneous fat being treated.

36. The method of claim 35 further including measuring the temperature of the subcutaneous fat being treated.

37. The method of claim 36 in which measuring the temperature of the subcutaneous fat being treated includes using a microwave radiometer.

38. The method of claim 37 in which the radiometer includes an antenna associated with the electrode.

39. The method of claim 38 in which the antenna is printed on the electrode surface contacting the patient's epidermis.

40. The method of claim 38 in which the electrode includes an opening and the antenna is located in the electrode opening.

41. The method of claim 36 further including determining a thermal dose as a function of the temperature of the subcutaneous fat being treated and the length of time the radio frequency energy is supplied to the electrode.

42. The method of claim 35 in which the electrode has a patent contact surface and further including adding an electrically non-conductive material on a periphery of the patient contact surface to limit the an electrically active portion of the patient contact surface.

43. The method of claim 35 further including adjusting the radio frequency energy supplied to the electrode to subject the subcutaneous fat to a thermal dose of between 0.1 and 10.0.

44. The method of claim 35 further including adjusting the radio frequency energy supplied to the electrode to subject the subcutaneous fat to a temperature of between 40° C. and 50′C.

45. The method of claim 35 further including supplying radio frequency energy to the electrode for between 10 and 30 minutes.

46. The method of claim 35 further including cooling the electrode.

47. A subcutaneous fat treatment system comprising:
an electrode for application to a patient's epidermis above subcutaneous fat to be treated;
a sensor for measuring the temperature of the subcutaneous fat to be treated;
a radio frequency source for applying radio frequency energy to the electrode; and
a controller subsystem, responsive to the sensor and controlling the radio frequency source and configured to:
control the radio frequency source to apply radio frequency energy to the electrode to reach and maintain a desired set subcutaneous fat temperature,
determine the thermal dose applied to the subcutaneous fat being treated, and
automatically adjust the radio frequency energy supplied to the electrode based on the measured temperature of the subcutaneous fat being treated and subjecting the subcutaneous fat to a thermal dose of between 0.1 and 10.0.

48. The system of claim 47 in which the sensor is a microwave radiometer.

49. A method of treating subcutaneous fat, the method comprising:
applying an electrode to a patient's epidermis above subcutaneous fat to be treated;
measuring the temperature of the subcutaneous fat to be treated;
applying radio frequency energy to the electrode;
controlling the radio frequency energy to reach and maintain a desired set subcutaneous fat temperature;
determining the thermal dose applied to the subcutaneous fat being treated; and
automatically adjusting the radio frequency energy supplied to the electrode based on the measured temperature of the subcutaneous fat being treated and subjecting the subcutaneous fat to a thermal dose of between 0.1 and 10.0.

50. The method of claim 49 in which measuring the temperature of the subcutaneous fat to be treated includes employing a microwave radiometer.

51. A subcutaneous fat treatment system comprising:
an electrode assembly for application to a patient's epidermis to produce a temperature change of the dermis and subcutis immediately underneath the electrode assembly;
the electrode having a patient contact surface;
the electrode having a cavity for receiving a cooling fluid;
a layer of electrically non-conductive material positioned on the periphery of the patient contact surface to limit the electrically active portion of the patient contact surface;
a radio frequency source for applying radio frequency energy to the electrode.

52. The system of claim 51 further including a controller configured to supply radio frequency energy level to the electrode and a cooling fluid temperature to produce a dermis temperature lower than the subcutis temperature.

53. The system of claim 52 in which the dermis temperature is below 37° C. and the subcutaneous temperature is between 40 and 500.
